(19) United States
(12) Patent Application Publication
(10) Pub. No.: US 2002/0012708 A1
(43) Pub. Date: Jan. 31, 2002

(54) USE OF ARTICHOKE (CYNARA) EXTRACTS

(75) Inventor: MICHEL O. RUEPP, REMSCHEID (DE)

Correspondence Address: BROBECK, PHLEGER & HARRISON, LLP, ATTN: INTELLECTUAL PROPERTY DEPARTMENT, 1333 H STREET, N.W. SUITE 800, WASHINGTON, DC, 20005, US

(73) Assignee: arr Pharma

(21) Appl. No.: 09/225,573

(22) Filed: Jan. 5, 1999

Related U.S. Application Data
(63) Continuation-in-part of application No. PCT/EP97/03561, filed on Jul. 5, 1997.

(30) Foreign Application Priority Data
Jul. 6, 1996 (DE) 196 27 376.5

(51) Int. Cl. A61K 35/78

(52) U.S. Cl. 424/725

(57) ABSTRACT

The present invention relates to a novel use of artichoke (Cynara) extracts, especially dry extracts, optionally in combination with Echinacea extracts and/or nettle (Urtica) extracts, for the preparation of medicaments, and to orally applicable medicaments.

In particular, the invention relates to the use of artichoke (Cynara) dry extracts, also in combination with Echinacea and/or Urtica extracts, for the preparation of medicaments for the treatment of diseases of the small intestine (damages from medicaments or infections), of the bone marrow (aplasia and insufficiency, for example, as a consequence of agranulocytosis caused by medicaments or radiation), thymus (dysfunction, aplasia or hypoplasia), spleen (dysfunction), lymph nodes (aplasia or hypoplasia due to damage from medicaments or radiation), for the adjuvant treatment, also in combination with chemopharmaceuticals, of analgesia, liver, pancreas and kidney diseases, of hypertension, of malignant tumors, especially of carcinomas of the mamma, cervix, colon or prostate gland. Cynara dry extracts are further suitable for cellular immunostimulation, for the therapy of leucocytopenia, granulocytopenia, lymphocytopenia, erythrocytopenia, and immunoglobulin deficiencies; in addition, it is suitable for bacterially or virally induced diseases, such as inflammatory diseases of the small intestine, pancreas and kidneys, hepatitis A, B and C, skin lesions (Ulcus cruris), Herpes simplex I and II, as well as Herpes zoster.

This application is a continuation-in-part of International Application PCT/EP97/03561, filed Jun. 6, 1997, designating the U.S., the text of which is incorporated herein by reference in its entirety.

The present invention relates to a novel oral use of artichoke (Cynara) extracts, optionally in combination with Echinacea extracts and/or nettle (Urtica) extracts, especially dry extracts.

Since about the 16th century, the wide-spread use of herbal books has provided a general knowledge of the utility of artichoke as a medicament.

In folk medicine, it is recommended for treating indigestions if caused by an underproductivity of bile, and it is a liver remedy and a cholagogue. The drug has been employed in the form of its aqueous or alcoholic extracts.

As a second field of action, a significant diuretic effect had been indicated, which laid the foundations of the plant's use as a dehydration agent as well. The associated increased elimination of uric acid salts by the increased diuresis is the reason for the drug's use as an antirheumatic agent. It has also been proposed as an antidiabetic in singular cases. As early as 1934, a compound, at the time chemically undefined, was isolated from artichoke leaves, and it was already referred to as the therapy-relevant component. This compound was identified as cynarin only in 1954. The whole extract evidently contains supplementary or synergistic substances. The whole extract may also be replaced by the uptake of the fresh plant. The active substance cynarin is not genuinely contained, but is produced in the course of the processing when the plant is boiled in aqueous media. As important components, there may be mentioned the three classes of substances, caffeoylquinic acids, bitter principles and flavonoids.

For pharmaceutical purposes, the large basal leaves are employed, possibly from the annual basal leaf culture. These provide the highest content of choleretically active components. The value of the drug is determined by the total of the caffeoylquinic acids, because the therapeutic principle of action of artichoke extracts is based on the total of the caffeoylquinic acids rather than on the isolated Cynara component cynarin alone. Good drugs contain at least 0.2% caffeoylquinic acids.

It has been the object of the present invention to provide novel medicaments and thus novel possible therapies by using artichoke extracts.

Accordingly, a first embodiment of the present invention is the use of artichoke (Cynara) extracts, especially dry extracts, for decreasing the serum levels of blood glucose, creatinin and/or bilirubin, for cellular immunostimulation, for the therapy of leucocytopenia, granulocytopenia, lymphocytopenia and of organ and tissue damages caused by infections and chemically, especially of the 0-MALT system of the small intestine, of the bone marrow, thymus, spleen, lymph nodes, liver, pancreas and kidneys.

Preferred embodiments can be seen from the dependent claims. According to the invention, defined parameters of the circulation and immune system and of defined functions of liver, pancreas and kidneys could be influenced in in-vivo studies with rats with a dry extract, in a galenic formulation as granules, from the fresh plant of artichoke leaves.

The test substance which was employed in an amount of 100 mg per kg of body weight induced:

a slightly negative deviating effect, as compared to the control group, on the average development of the body weight which is attributed to a diuretic effect;

no utilizable changes in the average organ weights of spleen and thymus;

an increase of the cell counts of leucocytes, polymorphonuclear granulocytes and lymphocytes, T, B, helper, suppressor and NK (natural killer) cells as a manifestation of cellular immunostimulation, wherein the dominant specific increase in B lymphocytes may be particularly pointed out;

a decrease of the levels of creatinin, bilirubin, urea, cholesterol, triglycerides, glucose and GPT.

The following are considered therapeutically desirable effects:

a decrease of the cholesterol and triglyceride levels in terms of a positive influence on pathological disorders of lipid metabolism and thus reduction of atherogenic risk factors;

a general increase of defined immunocompetent cells as a possible inspecific treatment of inflammatory processes of different causal pathogeneses;

a dominant specific increase of the cell counts of B lymphocytes as an adjuvant in the therapy of toxically initiated B cell suppression;

a reduction of the bilirubin level and of the liver-specific activity value of GPT under the aspect of a hepatocurative effect;

a decrease of the serum glucose level as a functional equivalent of a hypoglycemic effect;

a decrease of the serum creatinin and urea levels as an indication of an improvement of renal function and detoxification of the ammonia produced in protein metabolism, and in connection with this reaction cycle, an enhanced mitochondrial performance of the hepatocytes;

a lack of indications of undesirable effects.

The recorded changes of defined hematological and clinical-chemical analytical values are to be considered under two aspects:

The results observed in the parameters used according to the invention speak in favor, on one hand, of the hepatocurative, hepatoprotective and hypolipidemic effects, described in the literature, of the artichoke extracts employed, and on the other hand, of novel, as yet unknown, biological activities which could be utilized under therapeutic aspects in disorders of carbohydrate metabolism, liver and kidney functions, and deficiencies of the cellular immune state.

The orally administered artichoke extracts of various origins and concentrations exhibited bioequivalent potentials with respect to the increase of cell counts and the changes of the relative cell count values of all lymphatic cells. Bioequivalence was also found in the clinical-chemical parameters bilirubin, creatinin, GPT, cholesterol, calcium, potassium and protein.

According to the invention, it has been found that the artichoke (Cynara) extracts employed are particularly suitable for cellular immunostimulation. The use of the extracts caused an increase of the cell counts of leucocytes, polymorphonuclear granulocytes and lymphocytes, T, B, helper, suppressor and NK cells, especially B lymphocytes

The artichoke (Cynara) extracts are particularly useful for the treatment of disorders of carbohydrate metabolism, especially for improving the prediabetic metabolic condition, and for the dosage reduction of chemically defined antidiabetics including insulin, and for the simultaneous administration of the extracts when there is some residual function of the endocrinic pancreas. In addition to a treatment of liver and kidney functional disorders, the artichoke (Cynara) extracts are also useful for the adjuvant therapy of a disordered immune system as a consequence of endogenous and exogenous factors, for example, in chemo- or radiotherapy.

In addition, the artichoke (Cynara) extracts are also useful for the treatment of prediabetic, for example, senile forms, for the treatment of a latent diabetic metabolic condition, for example, due to pregnancy, infection, stress, obesity, and as an adjuvant therapy of Diabetes mellitus, when there is still some residual pancreatic function, in the form of a combination therapy.

In animal experiments, the Cynara preparation improves the morpho-functional substrate of the Langerhans islets in the pancreas in terms of an increased endocrinic pancreatic function. Both in animal experiments and clinically, an unambiguous hypoglycemic effect could be detected.

From a therapeutic point of view, this means, when there is still some residual pancreatic function:

reduction of the insulin dose (when 3×600-900 mg of the Cynara preparation/day is simultaneously administered, an individual decrease of the insulin quantity is required)

reduction of the chemically defined antidiabetics (individual dosage required); effects thereof: protective effect against the hepato-, nephro- and cardiotoxic potential of a chronical administration of chemically defined oral antidiabetics.

In order to further clarify the mechanism of action of the extract employed on the central metabolic organ, the liver, partial toxic alterations of the liver parenchyma were induced in vivo by a simultaneous administration of carbon tetrachloride. The liver necrosis caused by carbon tetrachloride was not prevented by the use of the artichoke (Cynara) extract according to the invention, but its extension was clearly limited. Perifocal, less damaged or undamaged parenchyma areas impressed due to a functional swelling of the nuclei, characterized by particularly large, bright nuclei with a loose chromatin structure and large, highly basophilic nucleoli.

These micromorphological indications of an increased protein biosynthesis of the intact liver cell areas between the nucleic necroses were coupled with an increased occurrence of liver cell mitoses and dinuclear or polynuclear hepatocytes. These findings can be interpreted in terms of a mobilization of the liver cell metabolism and a beginning liver cell regeneration, induced by the simultaneous treatment with the artichoke (Cynara) extracts.

This evaluation was supported by the detection of the qualitative and quantitative enzymatic activity of succinate dehydrogenase. Under the influence of the use of the artichoke extracts, a clear reduction of the succinate dehydrogenasenegative areas took place. The intact perifocal areas were characterized by a succinate dehydrogenase hyperactivity. The latter can be considered the manifestation of a compensatory hyperintensive reaction of the intact parenchyma.

In a further in-vivo study, the effect of artichoke extracts, also in combination with Echinacea extracts (E. pallida and E. angustifolia), on toxically induced alterations of the liver and lymphatic organs by cytostatics, especially cyclophosphamide, was examined. In rats, the latter substance induces atrophy of the red bone marrow, the lymphatic organs, Peyer's plaques, the thymus, the spleen and the lymph nodes. By a simultaneous treatment with Cynara extracts, the damaging effect of cyclophosphamide was reduced. By a further simultaneous treatment with Cynara and Echinacea extracts, the cell-damaging effects were even clearly reduced (synergistic effect of Cynara and Echinacea). After the administration of the alkylating cytostatic, a partial accidental regeneration can be histologically detected in the bone marrow, thymus, spleen, mesenteric and cervical lymph nodes and Peyer's plaques after simultaneous treatment with the Cynara preparation, which gets a synergistic positive influence from the simultaneous administration of Echinacea extracts. As to micromorphology, the corresponding organs show clear indications of a restoration of the organ-specific architecture and a clear repopulation of the reticular stroma with intact cells of the myeloid and especially the lymphatic group.

This accidental regeneration of the blood-forming and lymphoreticular tissues does not reveal any signs of an increased mitotic activity in terms of a pathologically increased proliferation. Cyclophosphamide induced an atrophy of the thymus in the form of an elimination of lymphocytes by an antiproliferative effect which was generally more pronounced in B cells than it was in T cells. These different effects of the lymphocytotoxic influence of cyclophosphamide is due to different metabolic performances of these cell qualities.

After simultaneous peroral administration of the artichoke extracts and cyclophosphamide to rats over a period of 14 days, a complete elimination of the lymphocytes from the cortex and marrow of the thymus could be prevented by the antitoxic potential of the artichoke extracts. This effect could even be increased by the additional administration of Echinacea extracts.

Already under the therapeutic influence of the artichoke extracts alone, a clear repopulation of the thymus with lymphocytes took place which was about 40% as compared to the controls.

In the case of the lymph node, for example, a repopulation of the reticular tissue with cells of the lymphatic group can be seen histologically. In the paracortical zone, densely packed intact lymphatic cells prevail, and in the cortical zone, there are loosely arranged intact lymphocytes with a clear tendency to form subcapsular follicles. With the restoration of the characteristic microstructure of the lymphoreticular tissue in the lymph node, its function evidently also returns.

Recently, the different response forms of the lymph node have gained practical importance: if a stimulation of the T cells (paracortical zone) is observed in the drainage area of carcinomas (carcinoma of the mamma, carcinoma of the cervix), there is a better prognosis for the patients than in the case where the B cell region or no region of the lymph node is activated.

A successful defense against living exogenous pathogens by the endogenous immune system is quite critically dependent on the strength and quality of the host's immune response. An intact cellular and humoral defense by the host organism can control the formation and spread of a tumor. However, the most important source of immunodeficiency today is the cytostatic corticoid therapy for cancer patients.

Ultimately, the conclusion can be drawn that the Cynara preparation, also in combination with Echinacea formulations, in oral administration is suitable as a therapeutic adjuvant for the treatment of malignant tumors as well as chemocytostatic and radiologic tumor treatment.

Although various secondary surrogates describe the pharmacodynamic activity profile in an extract-specific way, a “basic scheme of general reactions” can be seen in the activity.

Depending on the active substance, dosage and duration of the administration, target organs were influenced due to interactions between the endocrine, nervous and immune systems.

Similar effects on the O-MALT system of the small intestine, of the bone marrow, thymus, spleen, lymph nodes, liver, pancreas and kidneys as described above for Cynara and Echinacea were also experimentally observed in a rat animal model after the administration of Urtica extracts and Cynara extracts from the roots, the leaves and the herbage.

In the same study, artichoke extracts exhibited an identical therapeutic action on the mesenteric lymph node. In this organ as well, the complete mobilization under cyclophosphamide was compensated by a repopulation with lymphocytes under a treatment with artichoke extracts. This protective effect primarily concerned the lymphoblasts in the T regions, and in a less pronounced way the lymphocytes from the B regions and the mature microcellular lymphocytes from the T regions.

In a clinical outpatient study, the effect of the artichoke extracts was examined over a period of 14 weeks in patients suffering from hyperlipidemia and accompanying hepatopathy. The extracts had a positive influence on the pathological metabolic situation of the patient suffering from hypercholesterolemia and/or hypertriglyceridemia. At the same time, a reduction of increased liver-specific enzymatic activity values occurred.

### EXAMPLES

A clinical study on the effect of artichoke extracts in patients suffering from hyperlipidemia and hepatopathy was performed with 40 patients of the age of 50-80 years who were treated with the following composition for an average of 19 days:

dry extract from artichoke leaves (25-35:1) 60.00% by weight

extractant: purified water DAB 10

(DAB=German pharmacopoeia)

further ingredients:

| | | |
|---|---|---|
| glucose | 12.75% | by weight |
| highly disperse silica | 7.75% | by weight |
| talcum | 2.8% | by weight |
| magnesium stearate | 0.8% | by weight |
| calcium carbonate | 11.4% | by weight |
| saccharose | 0.6% | by weight |
| potato starch | 3.9% | by weight |

The patients were treated for existing disorders of lipid metabolism and hepatopathies while a medicamental permanent treatment of other basic diseases (diabetes, hypertension, cardiopathy, hyperuricemia, asthma, epilepsy, diseases of the thyroid gland and osteoporosis) which were simultaneously present was maintained unchanged.

Of this collective of patients, 28% had liver diseases, 12% were diabetics who obtained chemically defined antidiabetics, and 47% showed increased lipid and γ-GT values, in part with cardiac diseases, as well as hypertension. The patients suffering from cardiovascular diseases simultaneously continued to be treated with ACE inhibitors (45%), α-2 receptor antagonists (5%), Ca antagonists (40%), diuretics (45%) or with a corresponding combined preparation of these classes of substances (15%) during the phase of treatment with the Cynara preparation (300 mg).

It was remarkable that under the simultaneous intake of the Cynara preparation together with ACE inhibitors, α-2 receptor antagonists, Ca antagonists, diuretics or with a corresponding combined preparation of these classes of substances, the γ-GT values fell by 8.7%, the GPT values fell by 11.6%, and the GOT values fell by 10.0% after an average of 19 days.

In 21 patients with hyperlipidemic and hypertensive values, not only a reduction of the increased cholesterol and triglyceride values, but also a decrease of the systolic blood pressure values by an average of 5% and of the diastolic ones by 6.8% could be observed due to a lipid level reducing Cynara therapy. This allows to conclude that the Cynara therapy also has an antihypertensive effect in a limit value hypertensive condition, and in addition a synergistic adjuvant hypotensive effect after simultaneous administration with chemically defined antihypertensive agents for increased blood pressure values. Ultimately, this has the consequence that the necessary dosage of chemically defined antihypertensive agents can be reduced, and thus the risk of undesired side-effects can be limited.

It is known that, for example, the synthesis of the γ-GT in the liver can be induced by cholestase, chronical alcohol consumption and pharmaceuticals in therapeutic dosage. Both an increase in activity of the soluble γ-GT in the hepatocytes and a spread of the membrane-bound form occur. The increased γ-GT level to be measured in the serum after an induction of the synthesis depends on the kind and extent of the noxa.

These relationships, especially the increase of the γ-GT activity values, for example, due to a chronical administration of chemically defined pharmaceuticals, and, on the other hand, the reduction of increased γ-GT activity values due to the simultaneous administration of the Cynara preparations allow to conclude that the artichoke dry extracts, due to their simultaneous administration, do not enhance, but rather significantly reduce the hepatotoxic potential, pronounced in part, of the ACE inhibitors, α-2 receptor antagonists, Ca antagonists, diuretics or corresponding combined preparations.

These hepatoprotective and -curative effects of the Cynara preparation have been supported by results found in animal experiments in a simultaneous administration with clofibrate of the cyclophosphamides.

The clinical-chemical parameters used in the evaluation were essentially identical with those used in the present in-vivo studies. The results obtained in this patient study showed a remarkable similarity in a number of the parameters:

| | | | |
|---|---|---|---|
| total cholesterol | −12.0% | (patients) versus | −30% (in vivo) |
| GPT | −11.9% | versus | −11.6% |
| bilirubin | −7.7% | versus | −5.1% |
| glucose | −8.7% | versus | −8.5% |

It may be concluded therefrom that the reproducible data, collected in a methodologically precise manner, of the present in-vivo studies can altogether be transferred to the conditions in humans by way of an argument by analogy.

Male Wistar rats with an average body weight of 310 g were kept under conventional conditions at a room temperature of 21° C. +/−1° C., a relative humidity of about 60% and a 12 hour day/night rhythm. Before the experiment was begun, they were subjected to an acclimatization to the keeping conditions for 12 days.

The rats were fed with a pelletized standard diet, Altromin C1000.

The animals were given tap water ad libitum as the drinking water.

The artichoke extract and dosage employed were as follows:

1. Calcium carbonate and saccharide containing granules with a standardized dry extract from the fresh plant of artichoke leaves (25-23:1) and the above minor components;

extractant: purified water DAB 10;

starting drug: fresh artichoke leaves (Cynarae folium), Hagers Handbuch/PFX;

dosage form: granules 1.67 mg (≡1 mg of native Cynara dry extract, FC, suspended in 0.02 ml of dist. water), 0.5 ml=25 mg/250 mg of body weight.

Ten male Wistar rats were divided into the following treatment groups for the examination:

| | | Dosage | |
|---|---|---|---|
| | group | (mg/kg of body weight) | number of animals |
| I | control group | — | 5 |
| II | FC | 100 | 5 |

The test substance FC according to the invention was administered once a day to the non-sedated animals intragastrically by means of a rigid bulbous probe for a period of 7 days. The suspensions were freshly prepared immediately before the administration and administered in a homogeneous form. The control animals were given physiological saline in an equivalent volume.

The present studies aimed at simultaneously establishing, by means of a defined in-vivo model, specific markers which indicated early the therapeutic effect as a synergistic effect of differently influenced physiological feedback mechanisms.

In detail, there was detected in the peripheral blood:

combinedly:

erythrocytes (RBC), leucocytes (WBC), platelets (PLT), hemoglobin (HGB), hematocrit (HCT), erythrocyte indices:

mean corpuscular volume (MCV), mean corpuscular hemoglobin (MCH), mean corpuscular hemoglobin concentration (MCHC), and red cell distribution width (RDW)

by flow cytometry:

leucocyte differentiation: granulocytes, monocytes, lymphocytes, B cells, T cells, helper and suppressor cells

combinedly:

GPT, GOT;

glucose,

cholesterol, triglycerides

Na, Cl;

creatinin, urea.

Using the Sysmex K-1000 fully automated hematology analytic device, the following could be determined: WBC, RBC, PLT, HGB, HCT, MCV, MCH, MCHC. The main unit of this device essentially consisted of a hydraulic system (HS) and an electronic system (ES). The HS served to aspirate, pipette, dilute, mix and lyse. The ES analyzed and converted the signals of the HS and transmitted the results to a printer. Using microprocessors, the ES also monitored the test procedures and the test station and performed the quality control.

The hematocrit value indicated the percent volume fraction of erythrocytes in the blood. Hemoglobin, which is the chromoprotein contained in the erythrocytes, was an important cirterion for the diagnosis of anemias in addition to the erythrocyte count and the hematocrit value. Classification was performed through the erythrocyte indices. Erythrocyte size and hemoglobin content were characterized by the erythrocyte volume (MCV =mean corpuscular volume), the hemoglobin content of the erythrocytes (MCH =mean corpuscular hemoglobin), and the mean corpuscular hemoglobin concentration (MCHC). The red cell distribution width (RDW) was a measure of anisocytosis.

The parameters, such as enzymes, glucose, lipids, electrolytes, creatinin, urea and protein, were determined using the analytical device Cobas Mira in a selective, method-oriented, photometric or ion-selective way. The supplementary report provided data of quality control and statictics in a analysis-specific way.

The leucocyte differentiation was performed using the flow cytometer FACScan after appropriately lysing a whole blood sample (scattered light measurement).

The lymphocyte differentiation was performed after a specific monoclonal incubation by means of fluorescence-activated cell sorting (fluorescence measurement)

The following were quantitatively analyzed on the 7th treatment day:

leucocytes (total), lymphocytes (total),

T lymphocytes (CD2+/CD45 RA−),

B lymphocytes (CD2−/CD45 RA+),

helper lymphocytes (CD4−/CD8b+), and

NK cells (CD8a+/CD8b−).

For determining the phenotypes of the lymphocytes, they were incubated with the following antibodies supplied by Pharmingen, San Diego, U.S.A., to which a fluorochromeis coupled:

fluorescein isothiocyanate (FITC) conjugated mouse anti-rat CD2 monoclonal antibody,

R-phycoerythrin (R-PE) conjugated mouse anti-rat CD45RA OR A/B monoclonal antibody,

fluorescein isothiocyanate (FITC) mouse anti-rat CD4 monoclonal antibody,

R-phycoerythrin (R-PE) conjugated mouse anti-rat CD8 (ββ chain) monoclonal antibody,

fluorescein isothiocyanate (FITC) conjugated mouse anti-rat CD8a monoclonal antibody.

Approach:

a) CD2/CD45RA=T and B cells

b) CD4/CD8b=T₄ and T8 cells

c) CD8a/CD8b=NK cells

5 μl each of the antibodies was incubated with 50 μl of Na EDTA blood at room temperature in the dark for 20 min. The suspension was agitated with 2 ml of Lyses Reagent of Becton-Dickinson and incubated for 10 min as described. This was followed by a centrifugation at 400×g for 6 min, and the supernatant was poured off. The sediment was washed with 3 ml of Cell Wash and centrifuged at 400×g for 6 min. The pellet was taken up in 100 μl of Cell Wash. The suspension was analyzed using the flow cytometer.

During the acclimatization prior to the start of the experiment and throughout the duration of the experiment, the general condition of the animals was monitored. In addition, their body weights were determined daily.

The animals of all groups were sacrificed painlessly at the end of the experiment and dissected. The organs of the abdominal, pelvic and thoracic cavities were inspected macroscopically, and the spleen and thymus were weighed.

All animals had withstood the intragastric administration without impairment. The animals which had been treated with the test substance did not show any disadvantageous behavior clinically until the end of the experiment. During the treatment period, the average body weight increased in the groups as follows:

I control group by 12.6%

II FC by 8.1%.

The average body weight of the treatment group FC increased by a lesser percentage as compared to that of the control group. This was predominantly due to the aquaretic effect of the test substance.

The potential of the test substance FC was determined indirectly through its stimulating effect on the leucocyte (WBC), erythrocyte (RBC) and platelet (PLT) counts.

Table 1 contains the individual measured values of these cell counts after the end of the treatment, and Table 2 shows the corresponding average cell counts per group and the corresponding percent change of these values as compared to those of the control group.

The following was measured for the test substance FC according to the invention:

a clear increase in leucocyte count by 35%¹⁾;

the other parameters, such as RBC and PLT, varied within a physiological range of from −1% to −2%. (¹⁾ corresponding values of the control group=100%)

TABLE 1

| | WBC | RBC | HGB | HCT | MCV | MCH | MCHC | PLT |
|---|---|---|---|---|---|---|---|---|
| group I | | | | | | | | |
| Average | 8820 | 5.954 | 7.74 | 0.3712 | 62.38 | 1301.2 | 20.86 | 1049.8 |
| Standard deviation | 617.74 | 0.20 | 0.19 | 0.01 | 1.40 | 47.94 | 0.43 | 41.37 |
| group II | | | | | | | | |
| Average | 11.920 | 5.834 | 7.86 | 0.3696 | 63.34 | 1347.6 | 21.26 | 1038.25 |
| Standard deviation | 1410.53 | 0.09 | 0.14 | 0.01 | 1.59 | 24.79 | 0.21 | 87.01 |

Erythrocyte count (RBC) in 10¹²/1, leucocyte (WBC) and platelet (PLT) counts in 10⁹/1. Molar concentration of hemoglobin (HGB) in mmol/l, hematocrit (HCT) in 1/1, MCV in fl, MCHC in mmol/l, MCH in amol.

(conversion:mmol/l g/dl×0.6206;

1/1%×100; fl=μm³; fmol=pg×0.062;

amol=62×pg)

| group | mg/kg of body weight/day |
|---|---|
| I control | — |
| II FC | 100 |

TABLE 2

| | WBC | RBC | HGB | HCT | MCV | MCH |
|---|---|---|---|---|---|---|
| group I | 8820 | 5.954 | 7.74 | 0.3712 | 62.38 | 1301.2 |
| group II | 11,920 | 5.834 | 7.86 | 0.3696 | 63.34 | 1347.6 |

change with respect to group I (%)

| | WBC | RBC | HGB | PLT | HCT | MCV |
|---|---|---|---|---|---|---|
| group II | 35.15 | −2.02 | 1.55 | −1.10 | −0.43 | 1.54 |

Average erythrocyte cell counts in 10 /1, leucocyte and platelet counts in 10 /1, Molar concentration of hemoglobin in mmol/l, hematocrit in 1/1, MCV in fl, MCH in amol, MCHC in mmol/l.

Percent changes of the stated values as compared to the corresponding values of the control group.

| group | mg/kg of body weight/day |
|---|---|
| I control | — |
| II FC | 100 |

The test substance FC does not show any physiologically important influence on HGB, HCT, MCV and MCHC.

The following clinical-chemical metabolic measuring quantities were analyzed in a combined and method-specific manner:

bilirubin, creatinin, urea, glutamate oxalacetate transaminase (GOT), glutamate pyruvate transaminase (GPT), γ-glutamyl transferase (GGT), glucose, cholesterol, triglycerides, calcium and sodium.

For the test substance FC, there was measured:

a clear reduction of:

creatinin by 16.7%

GPT by 11.6%

cholesterol by 30.0%

glucose by 8.5%

a slight reduction of:

bilirubin by 5.1%

urea by 4.6%

GLDH by 3.2%

triglycerides by 1.0%

For the test substance FC, bioequivalent changes of bilirubin, creatinin, GPT, cholesterol, calcium, potassium and protein were measured which were comparable in nature and intensity.

In the treatment group II, the cell counts of the polymorphonuclear granulocytes, monocytes, T and B lymphocytes, helper, suppressor and NK cells were represented as compared with the control group.

For the test substance FC, there was measured an increase of:

leucocytes by 35.2%¹⁾

lymphocytes by 31.1%

T lymphocytes by 15.3%

B lymphocytes by 58.5%

helper cells by 14.0%

suppressor cells by 20.3%

monocytes by 24.5% (¹⁾ corresponding values of the control group=100%)

The present invention provides improvements in therapeutic effectiveness for various established therapies, the improvement consisting of co-administration of artichoke extracts (extracts of Cynara sp.) as an adjunct to the established therapy. Optionally, combination adjunctive therapy, in which extracts of artichoke are administered along with extracts of Echinacea or nettles (Urtica), is also provided. Adjunctive therapy according to the present invention is of particular value for the treatment of organ and tissue damages caused chemically or by radiation or infections, especially of the 0-MALT system of the small intestine (inflammatory diseases, Enteritis regionalis Crohn), of the bone marrow (bone marrow aplasia), thymus (dysfunction, aplasia or hypoplasia), spleen (dysfunction) and the lymph nodes (aplasia or hypoplasia due to damage from medicaments or radiation).

Adjunctive therapy according to the present invention is also of particular value for the treatment of organ and tissue damages of the liver (atrophy, necrosis, hepatitis A, B and C), pancreas (insufficiency of the exocrinic secretory function of proteases, esterases, carbohydrases and nucleases, and the endocrinic functional disorder of carbohydrate metabolism [Langerhans islets]) and the kidneys (insufficiency) . Adjunctive therapy according to the present invention is likewise of particular value for the treatment of general immunesuppressed conditions, for cellular immunostimulation, for the therapy of leucocytopenia, granulocytopenia, lymphocytopenia, and in immunoglobulin deficiencies.

In one mode, the adjunctive therapy of the present invention provides the cellular immunostimulation which causes an increase of the cell counts of leucocytes, polymorphonuclear granulocytes and lymphocytes, T, B, helper, suppressor and NK cells, especially B lymphocytes, in the terminal vascular system. Thus, upon therapy according to the present invention, an increase in vital lymphocytes can be seen in the cortical zone and follicles in Peyer's plaques (small intestine) which is associated with a secretory induction of sIGA into the small intestine, an increase of all blood forming elements can be seen in the bone marrow, a cellular stimulation of immunocompetent cells can be detected in the thymus, spleen and mesenteric lymph nodes, and/or the metabolic functions of the liver, pancreas and kidneys (reduction of the creatinin serum levels) are clearly activated.

In another mode, the present invention provides improvement, by administration of artichoke extracts, to methods for the treatment of disorders of carbohydrate metabolism and of liver and kidney functions, for the treatment of prediabetes, especially senile forms, for the treatment of infections, stress and/or obesity, or for the treatment of blood hypertension in a limit value hypertensive condition, or by use of artichoke extracts as synergistic adjuvants after simultaneous administration with other chemically defined antihypertensive agents.

In yet another mode, the present invention provides for the use of artichoke extracts as oral adjuvants for the treatment of malignant tumors, especially for the treatment of carcinomas of the mamma, cervix, colon or prostate gland, also in combination with cytostatics, especially cyclophosphamide, or radiation therapy. In particular, administration of artichoke extracts according to this invention can increase the tolerance of chemotherapy, cytostatic or radiologic therapies, especially in combination with chemically defined substances selected from clofibrate, ACE inhibitors, α-2 receptor antagonists, Ca antagonists and/or diuretics. The artichoke extract of this invention is thus effective as an analgetic adjuvant, especially as a pharmacological component having a positive influence on the interactions between the endocrine, nervous and immune systems.

In another mode, this invention provides for use of artichoke as an adjuvant for the treatment of bacterially or virally induced diseases, especially inflammatory diseases of the small intestine (Enteritis regionalis Crohn), pancreas and kidneys, as well as liver atrophy, hepatitis A, B and C, skin lesions (Ulcus cruris), and also Herpes simplex I and II, and Herpes zoster.

The present invention provides for improved therapeutic efficacy based on administration of artichoke (Cynara) extracts. The artichoke extract may be obtained a fresh plant, especially extracted juice as extracted using aqueous, organic and/or supercritical solvents, especially carbon dioxide, in a dried form, especially powder as dry extracts from the fresh plant or drug (dried plant), as granules, especially capsule, and/or as a tablet, especially coated tablet, or pastille. Typically, Cynara extracts with a drug-to-extract ratio (DEV native, i.e., the weight ratio of plant matter to extractant) of from 25:1 to 35:1 are employed. In a preferred mode, artichoke (Cynara) dry extracts are administered as an orally applicable medicament in the form of calcium carbonate and saccharide containing granules. Optionally, artichoke (Cynara) extracts are employed in combination with Echinacea extracts, especially dry extracts (E. pallida and E. angustifolia), and/or with nettle (Urtica) extracts, especially dry extracts from the roots, leaves or herbage. Typically Echinacea extracts with a drug-to-extract ratio of from 4:1 to 8:1 are employed and/nettle (Urtica) extracts with a drug-to-extract ratio of from 6:1 to 15:1 are employed. The dose of artichoke extract may be from 1 to 10,000 mg per kg body weight. As an initial dose, the extract may be administered to provide the biological equivalent of 100 mg (based on dry matter in the native extract) per kg of body weight. In clinical trials, the following doses of dry extract (native dry extract, excluding any other ingredients) have been administered:

preclinical:

100 mg dry extract/kg body weight per day (experimental data with rats)

clinical:

900 mg/day/patient 15 mg/kg body weight per day

1800 mg/day/patient 30 mg/kg body weight per day

2700mg/day/patient 45 mg/kg body weight per day

3600mg/day/patient 60 mg/kg body weight per day

Based these doses, the skilled clinician will, as a matter of routine, optimize the dose for the individual patient by monitoring the effects described herein for the particular therapeutic application, which may include increasing the dose in certain cases.

1. Use of artichoke (Cynara) extracts, especially dry extracts from the fresh plant, for the preparation of medicaments for the treatment of organ and tissue damages caused by radiation, infections and chemically, especially of the O-MALT system of the small intestine (inflammatory diseases, Enteritis regionalis Crohn), of the bone marrow (bone marrow aplasia), thymus (dysfunction, aplasia or hypoplasia), spleen (dysfunction) and the lymph nodes (aplasia or hypoplasia due to damage from medicaments or radiation), liver (atrophy, necrosis), hepatitis A, B and C, pancreas (insufficiency of the exocrinic secretory function of proteases, esterases, carbohydrases and nucleases, and the endocrinic functional disorder of carbohydrate metabolism [Langerhans islets]) and the kidneys (insufficiency), and of general immunesuppressed conditions, for cellular immunostimulation, for the therapy of leucocytopenia, granulocytopenia, lymphocytopenia, and in immunoglobulin deficiencies.

2. The use according to claim 1, characterized in that the cellular immunostimulation causes an increase of the cell counts of leucocytes, polymorphonuclear granulocytes and lymphocytes, T, B, helper, suppressor and NK cells, especially B lymphocytes, in the terminal vascular system, that an increase in vital lymphocytes can be seen in the cortical zone and follicles in Peyer's plaques (small intestine) which is associated with a secretory induction of sIGA into the small intestine, that an increase of all blood forming elements can be seen in the bone marrow, that a cellular stimulation of immunocompetent cells can be detected in the thymus, spleen and mesenteric lymph nodes, and/or that the metabolic functions of the liver, pancreas and kidneys (reduction of the creatinin serum levels) are clearly activated.

3. The use according to claim 1 or 2 for the treatment of disorders of carbohydrate metabolism and of liver and kidney functions, for the treatment of prediabetic, especially senile forms, for the treatment of infections, stress and/or obesity, for the treatment of blood hypertension in a limit value hypertensive condition, and as a synergistic adjuvant after simultaneous administration with chemically defined antihypertensive agents.

4. The use according to claim 1 or 2 as oral adjuvants for the treatment of malignant tumors, especially for the treatment of carcinomas of the mamma, cervix, colon or prostate gland, also in combination with cytostatics, especially cyclophosphamide, or radiation therapy.

5. The use according to claim 1 for increasing the tolerance of chemotherapy, cytostatic or radiologic therapies, especially in combination with chemically defined substances selected from clofibrate, ACE inhibitors, α-2 receptor antagonists, Ca antagonists and/or diuretics, as an analgetic adjuvant, especially as a pharmacological component having a positive influence on the interactions between the endocrine, nervous and immune systems.

6. The use according to claim 1 or 2 as an adjuvant for the treatment of bacterially or virally induced diseases, especially inflammatory diseases of the small intestine (Enteritis regionalis Crohn), pancreas and kidneys, liver atrophy, hepatitis A, B and C, skin lesions (Ulcus cruris), and also Herpes simplex I and II, and Herpes zoster.

7. The use according to one or more of claims 1 to 6 as a fresh plant, especially extracted juice as extracted using aqueous, organic and/or supercritical solvents, especially carbon dioxide, in a dried form, especially powder, dry extracts from the fresh plant or drug (dried plant), granules, especially capsule, and as a tablet, especially coated tablet, or pastille.

8. The use according to one or more of claims 1 to 7, characterized in that Cynara extracts with a drug-to-extract ratio (DEV native) of from 25:1 to 35:1 are employed.

9. The use according to one or more of claims 1 to 8, characterized in that said artichoke (Cynara) extracts are employed in combination with Echinacea extracts, especially dry extracts (E. pallida and E. angustifolia), and/or with nettle (Urtica) extracts, especially dry extracts from the roots, leaves or herbage.

10. The use according to claim 9, characterized in that Echinacea extracts with a drug-to-extract ratio of from 4:1 to 8:1 are employed.

11. The use according to claim 9, characterized in that nettle (Urtica) extracts with a drug-to-extract ratio of from 6:1 to 15:1 are employed.

12. An orally applicable medicament containing artichoke (Cynara) dry extracts as defined in one or more of claims 1 to 11 in the form of calcium carbonate and saccharide containing granules.
